(19) United States
(12) Patent Application Publication
(10) Pub. No.: US 2021/0322599 A1
(43) Pub. Date: Oct. 21, 2021

(54) AUTOMATED SURFACE DISINFECTING SYSTEM FOR INTERACTIVE TERMINALS

(71) Applicants: Joseph Patrick Schmit, Las Vegas, NV (US); Kristina Diane Schmit, Las Vegas, NV (US)

(72) Inventors: Joseph Patrick Schmit, Las Vegas, NV (US); Kristina Diane Schmit, Las Vegas, NV (US)

(21) Appl. No.: 17/234,985

(22) Filed: Apr. 20, 2021

Related U.S. Application Data
(60) Provisional application No. 63/012,788, filed on Apr. 20, 2020.

(51) Int. Cl. A61L 2/10; A61L 2/24; G07F 17/32

(52) U.S. Cl. CPC A61L 2/10; A61L 2/24; A61L 2209/12; A61L 2209/111; G07F 17/3216

(57) ABSTRACT

Systems and methods for disinfecting surfaces of interactive terminals having one or more components intended for user interaction therewith which includes a lamp for supporting an ultraviolet light source for directing ultraviolet light in the direction of the user interactive components when the interactive terminal is not in use.

### CROSS-REFERENCE TO RELATED APPLICATIONS

This application claims the priority benefit of U.S. Provisional Patent Application No. 63/012,788 filed Apr. 20, 2020, the disclosure of which is incorporated herein by reference.

### BACKGROUND OF THE INVENTION

The invention is directed to systems and methods for disinfecting interactive terminals, which as used herein, includes any computerized platform having components designed for one or more users to interact therewith, such as automated teller machines, point of service terminals, kiosks, arcade game machines and electronic gaming machines, each of which include one or more components that are intended for, and responsive to, user interaction.

Many government agencies, business operators and merchants offer interactive terminals to carry out commercial transactions, such as banking, governmental and retail transactions. Interactive terminals, such as automated teller machines, are often provided for customer convenience, whereas in other instances, the interactive terminals themselves are a critical source for generating revenue. For example, electronic gaming machines, are important sources of revenue in the casino industry.

Interactive terminals are generally openly available for use by multiple customers in succession, and are often adjacent one another due to space constraints, or for other reasons, such as for example, ease in installation, operation, management and power supply. Use of interactive terminals generally involves physical contact over a period of time or session. Even if physical contact is eliminated, during the session the user comes within close proximity of the interactive terminal.

There are current and persisting concerns about the spreading of infectious agents through the ability of infectious agents such as COVID-19 to remain infectious on surfaces of inanimate objects for an extended period of time. This method for infectious agents to spread is of great concern with respect to the normal usage of interactive terminals.

It has been well established that UV light has germicidal properties. Specifically, the mechanism by which UV light kills microorganisms is by damaging the genetic material, the deoxyribonucleic acid (DNA), of the microorganisms. DNA, specifically has a maximum absorbency of UV light at 253.7 nm. UV light is separated into 3 distinct categories: UV-A (315-400 nm), UV-B (280-315 nm), and UV-C (200-280 nm). Far-UV-C is a form of ultraviolet light having a narrow band of wavelengths, that is, approximately 207-222 nm, which is short enough to prevent damage to living human cells while still being able to penetrate and kill viruses and bacteria on surfaces.

There is a need in the art for systems and methods which limit the potential spread of infectious agents through the use of such interactive terminals which incorporate components which are intended for user interaction.

### SUMMARY OF THE INVENTION

The invention is generally directed to systems and methods for resolving the issues cited above, among other things.

Some embodiments of the invention are directed to a system for disinfecting the surface of a gaming machine, the gaming machine including front and back surfaces, the front surface including a console area, the console area having one or more components intended for user interaction therewith, the system comprising: a lamp mounted adjacent to the console area, the lamp including a housing for supporting an ultraviolet light source therein, the ultraviolet light source being configured to emit ultraviolet light in the range of about 207 nanometers to about 222 nanometers, the housing restricting the transmission of ultraviolet light emitted from the ultraviolet light source in all directions except for in the direction of the one or more components intended for user interaction therewith, wherein illumination of the light source is responsive to detection of a treatment actuating event; and a processing device operatively associated with a memory, the lamp and the gaming machine, wherein the processing device is configured for detecting a treatment actuating event stored in the memory and causing the illumination of the light source responsive to detecting the treatment activating event, wherein the illumination of the light source is deactivated by the processing device responsive to detecting a treatment deactivating event stored in memory.

In some embodiments, the treatment activating event stored in the memory comprises a SAS protocol signal.

In some embodiments, the treatment deactivating event stored in the memory comprises a SAS protocol signal.

In some embodiments, the system further comprises a motion detector configured to detect the user being positioned adjacent to the console as a treatment deactivating event.

In some embodiments, the housing encloses the ultraviolet light source on all sides except for one side, the one side comprising an opening, opposing side lips and opposing end lips configured to direct ultraviolet light from the ultraviolet light source through the opening and on to the one or more components intended for user interaction.

Some embodiments of the invention are directed to a method for disinfecting the surface of a gaming machine, the gaming machine including front and back surfaces, the front surface including a console area, the console area having one or more components intended for user interaction therewith, the system comprising the steps of: detecting a treatment activating event associated with the gaming machine, wherein the treatment activating event is associated with no user activity at the gaming machine; activating a lamp mounted adjacent to the console area responsive to the detection of the treatment activating event, the lamp including a housing for supporting an ultraviolet light source therein, the ultraviolet light source being configured to emit ultraviolet light in the range of about 207 nanometers to about 222 nanometers, the housing restricting the transmission of ultraviolet light emitted from the ultraviolet light source in all directions except for in the direction of the one or more components intended for user interaction therewith, wherein activating the lamp illuminates the ultraviolet light source; detecting user activity at the gaming machine; deactivating the lamp responsive to the detection of user activity at the gaming machine; and repeating steps a) through d).

In some embodiments of the aforementioned method, the step of detecting a treatment activating event further comprises detecting signals generated within the gaming machine through a communication device in communication with the gaming machine.

In some embodiments of the aforementioned method, the step of detecting a treatment activating event further comprises receiving data from a motion detector configured for detecting motion or the lack thereof in proximity to the gaming machine.

### BRIEF DESCRIPTION OF THE DRAWINGS

Various aspects of the present invention are illustrated by way of example, and not by way of limitation, in the accompanying drawings, wherein:

FIG. 1 is a perspective view of an ultraviolet light disinfecting treatment system of the invention incorporated with an exemplary gaming machine;

FIG. 2 is a perspective view of another ultraviolet light disinfecting treatment system of the invention incorporated with the exemplary gaming machine;

FIG. 3 is a perspective schematic view of an ultraviolet light disinfecting treatment system of the invention incorporated with a wall-mounted automated teller machine;

FIG. 4 is a front view of an ultraviolet light disinfecting treatment system of the invention incorporated with a standalone automated teller machine;

FIG. 5a is a perspective view of a housing with an ultraviolet light source therein used in the exemplary embodiment shown in FIG. 1;

FIG. 5b is a schematic view of one side of the housing shown in FIG. 5a having an opening therein for enabling the transmission of ultraviolet light from the ultraviolet source within the housing; and

FIG. 6 is flow chart of a method of providing a disinfecting treatment in accordance with some embodiments of the invention.

### DETAILED DESCRIPTION OF SOME EMBODIMENTS OF THE INVENTION

In some embodiments, the system of the invention is directed to an automated interactive terminal disinfecting system and method comprising a communication device in communication with a controller for an interactive terminal, the interactive terminal including a user-facing surface, the communication device receiving signals from the controller indicative of an initiation of a session of activity at the interactive terminal and a termination of the session of activity; a processing device for monitoring signals received by the communication device relating to the initiation and the termination of the session of activity and determining the initiation of the session of activity and the termination of the session of activity; a UV light lamp mounted on the interactive terminal and configured to emit Far UV Light (defined herein as UV light having a wavelength within a range of about 207 nm to about 222 nm and any other viral bacterial eradicating wavelength that is considered safe for human contact and use as described herein) to illuminate the user-facing surface of the interactive terminal, wherein the UV light lamp is activated for illumination for a preset period of time responsive to the processing device determining the termination of the session of activity and deactivated for illumination responsive to one of the termination of the preset period of time and the processing device determination of the initiation of a subsequent session of activity.

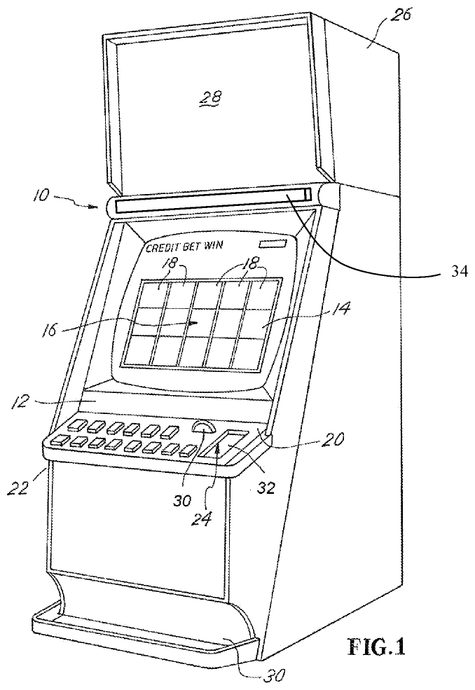

In FIG. 1, reference numeral 10 generally designates a gaming machine including components for operating a game thereon. The machine 10 includes a console 12 having a video display unit 14 on which a game 16 is played, in use. Game 16 may be a spinning reel game which simulates the rotation of a number of spinning reels 18. It will, however, be appreciated that the invention is equally applicable to other types of games such as card games, pachinko or any type of gambling game. The machine 10 includes a top box 26 on which artwork 28 is carried. The artwork 28 includes paytables, details of bonus awards, etc. A customer front interface midtrim 20 of the machine 10 houses a bank 22 of buttons for enabling a player to play the game 16. The midtrim 20 also houses an input device 24 and a player tracking card reader 30. Input device 24 can include currency and ticket reading/printing device 32.

In some embodiments, systems and methods of the invention provide a ultraviolet (UV) illumination device or lamp 34 which is mounted above console 12 of gaming machine 10 and angled, positioned or otherwise configured for directing UV-C light onto various surfaces of the gaming machine.

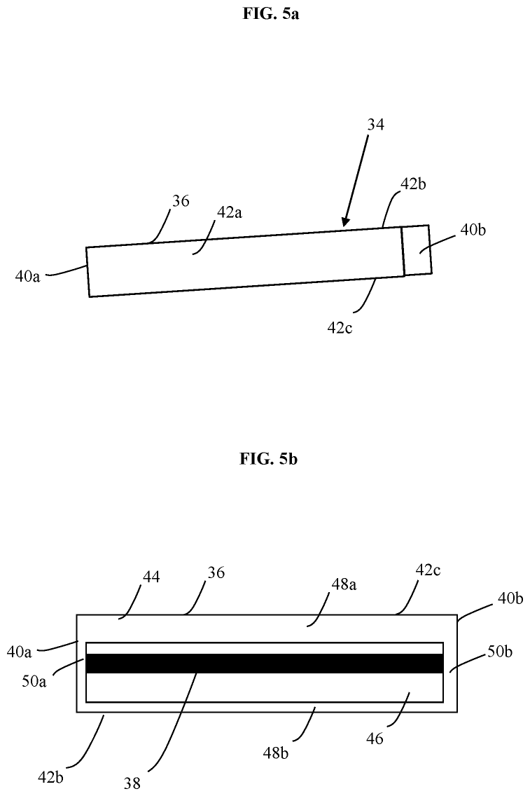

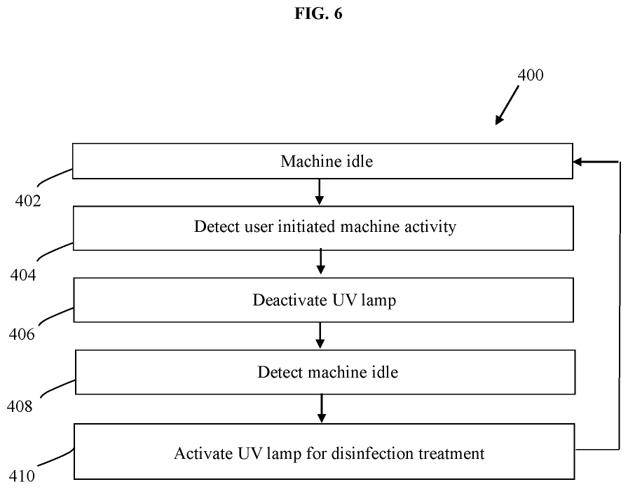

For example, lamp 34 may include a housing made of a non-transparent or opaque material, such as PVC, which is configured to limit or restrict UV light in all directions except for the direction of the user interactive components of gaming machine 10, such as bank 22. An exemplary embodiment of lamp 34 is shown in FIGS. 5a and 5b, which includes a housing 36 for supporting a UV light source 38 therein connected to a power source, such as a battery or may be connected to machine 10 for both power and purposes of detecting machine activity as described in FIG. 6. Housing 36 in this embodiment is substantially rectangular and encloses UV light source 38 on both ends 40a,b and sides 42a,b,c. In this embodiment, sides 42a,b,c and ends 40a,b are non-transparent and substantially enclose light source 38 so as to restrict transmission of light therethrough. Side 44 of housing 36 includes an opening 46 defined by opposing inner side lips 48a,b and inner end lips 50a,b. Opposing side lips 48a,b and end lips 50a,b of side 44 are configured so that opening 46 is dimensioned and positioned so that the desired user-interactive components of machine 10 are exposed to UV light from source 38 while light from source 38 is restricted from being emitted in other directions. As shown in this embodiment, lips 48a and 50b extend further over opening 46 than lips 48b and 50a to restrict and direct UV light from source 38 as desired.

In some embodiments, UV lamp 34 may be an UV LED light, for producing UV-C or Far-UV-C light, and targeting that light at various surfaces of a gaming machine, such as surfaces mostly likely to experience a physical interaction with a person or be within a physical range of a person to possibly enable the transmission of an infectious agent. For example, UV lamp 34 may be directed to target display unit 14 and midtrim 20, including bank 22 and input device 24 and player tracking card reader 30.

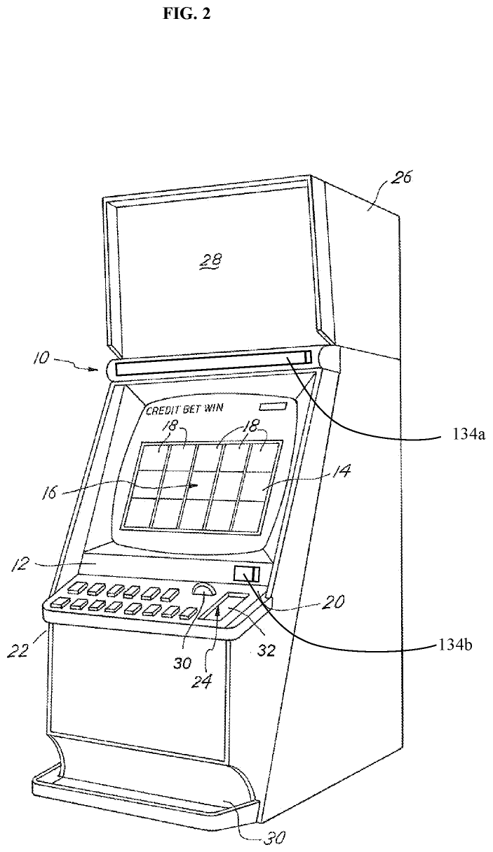

In some embodiments, there are more than one UV lamps 34 mounted on a gaming machine such as gaming machine 10. For example, as shown in FIG. 2, a UV lamp is mounted adjacent to player tracking card reader 30 and configured to target UV light on midtrim 20. As shown in FIG. 2, a UV lamp 134a is mounted above console 12 to target display unit 14 and a UV lamp 134b is mounted adjacent to player tracking card reader 30 to target midtrim 20.

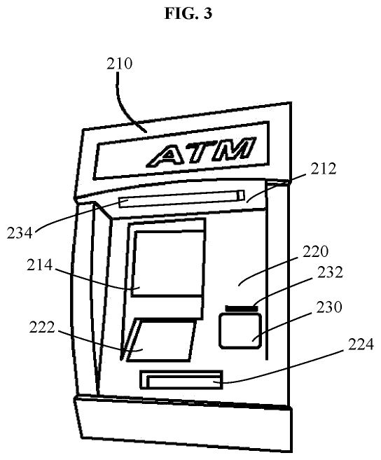

It should be readily apparent that the systems and methods of the invention may be deployed in other apparatus which are configured for physical interaction with multiple users. FIG. 3 depicts an automated teller machine (ATM) 210 which may be mounted on a wall or column. ATM 210 includes a console 212 with display 214, and various peripherals on a front customer interface 220. Customer interface 220 of ATM 210 includes a bank 222 of buttons, a deposit receiving device 224, a card reader 230, and a receipt printer 232. A UV lamp 234 is mounted above console 212 to target display 214 and customer interface 220, including the aforementioned peripherals.

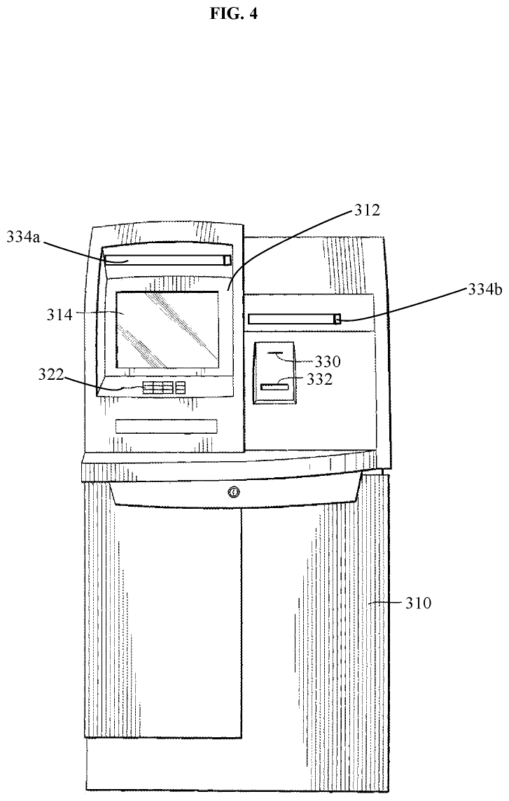

FIG. 4 depicts an ATM 310 which may be a standalone device or terminal, including a console 312, a display 314 and bank 322 of buttons. A first UV lamp 334a is mounted on the console 312 above the display 314 and configured to direct UV light on the surface of display 314 and bank 322. ATM 310 also includes a second UV lamp 334b which is mounted adjacent to the card reader 230 and receipt printer 332 and configured to direct UV light thereon.

In some embodiments, the systems and methods of the invention are actuated on demand, periodically or automatically upon certain triggering events. For example, a processing device associated with the UV lamp of the invention or any interactive terminal, such as a gaming machine or ATM, such as gaming machine 10 or 110 or ATM 210 or 310, can be configured to detect certain gaming machine and ATM events, compare detected events with a preset criteria for actuating the UV lamp, and trigger the UV lamp responsive to detecting an event which satisfies the preset criteria. For example, an event can be the insertion of currency or physical item such as a ticket or ATM card. Events may also include data received from sensing devices such as motion detectors.

FIG. 6 illustrates an exemplary method of the invention generally designated by reference numeral 400. As indicated by block 402, the machine, which may be any interactive terminal having components in which multiple users interact, including a gaming machine or ATM, which further includes a UV lamp directed at one or more components of the machine which would normally be physically contacted by a user is idle, such as touchscreen display and button panel. For purposes of this description, the term “idle” refers to a mode of operation or a status in which a user is not interacting with the machine without limitation to other machine activity, such as internal processing operations and the operation of the UV lamp. As shown by block 404, user initiated machine activity is detected and the UV lamp, if activated, is deactivated as indicated by block 406. For purposes of this embodiment user initiated machine activity includes any interaction with a user of the machine, including but not limited to, inserting a card or ticket in a ticket or card reading device, pressing a button on a button bank, and touching of a touchscreen, as well as detection of motion, if the machine is so configured, such as the user sitting down at a gaming machine seat or being positioned within a certain proximity of the machine.

As shown by block 408, the system and method of the invention is configured to detect machine idle, which may be indicative of a user no longer initiating machine activity and/or motion no longer being detected by a user positioned within a certain proximity of the machine. As shown by block 410, the UV lamp of the invention is activated to provide a disinfecting treatment.

In some embodiments, the machine may be configured to institute a countdown, which may be displayed on a display screen, communicating the timing upon which the treatment process is to begin. In some embodiments, the machine is configured to communicate that the treatment process is initiated and provide a countdown indicating when the process will be completed, upon which the machine returns to the idle status of block 402 until detecting user initiated activity in block 404.

In some embodiments, the system of the invention may be in communication with the machine for listening to internal signals generated or transmitted by machine components, and configured to detect certain signals as indicative of machine activity or idle status. For example, the system of the invention as shown in FIG. 1 may be configured to have a communication connection with the gaming machine controller, motherboard, slot machine interface board (SMIB) or other gaming machine component, for detecting signals, such as SAS protocol signals indicative of credit meter status, bill meter status, machine idle time, cash-out or ticket printing commands, which is further indicative of the machine returning to an idle status. In other words, the system of the invention detects when credit is zero, such as when all credit is lost or a user retrieves remaining credit stored in the credit meter. The system may begin a countdown thereafter before activating the UV lamp in block 410 to ensure that no further user-initiated activity is occurring prior to the start of a treatment cycle. The user-initiated activity detected in block 404 may also be an internal signal, such as the SAS protocol signal for receiving credit to the credit meter or card reader activation responsive to receiving a player loyalty card therein. The communication connection may be a wired or wireless connection via a communication device associated with the system of the invention.

In some embodiments the UV lamp may be continuously activated or operate pursuant to demand or an integrated scheduling application, which may reside either in a processor or computing device associated with the UV lamp or interactive terminal, until the UV lamp is deactivated pursuant to user-initiated activity being detected in block 404.

Some embodiments also relate to an apparatus for performing the operations herein. Such an apparatus may be specially constructed for the purposes, e.g., a specific computer, or it may comprise a general-purpose computer selectively activated or reconfigured by a computer program stored in the computer. Such a computer program may be stored in a computer-readable storage medium such as, but not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions with each coupled to a computer system bus. Memory can include any of the above and/or other devices that can store information/data/programs and can be a transient or non-transient medium, where a non-transient or non-transitory medium can include memory/storage that stores information for more than a minimal duration. Furthermore, the computers referred to in the specification may include a single processor or may be architectures employing multiple processor designs for increased computing capability.

The algorithms and displays presented herein are not inherently related to any particular computer or other apparatus. Various general-purpose systems may also be used with programs in accordance with the teachings herein, or it may prove convenient, or in some instances necessary, to construct more specialized apparatus to perform the method steps. Those skilled in the art will readily appreciate that the apparatus described herein may include various computer and network related software and hardware, such as programs, operating systems, memory storage devices, data input/output devices, data processors, servers with links to data communication systems, wireless or otherwise, which may be specific and specialized for the purposes described herein and for other purposes, such as maintaining a secure terminal environment.

The order of execution or performance of the operations in the embodiments of the invention illustrated and described herein is not essential, unless otherwise specified. That is, the operations described herein may be performed in any order, unless otherwise specified, and embodiments of the invention may include additional or fewer operations than those disclosed herein. For example, it is contemplated that executing or performing a particular operation before, contemporaneously with, or after another operation is within the scope of aspects of the invention.

While exemplary apparatus, systems and methods of the invention have been described herein, it should also be understood that the foregoing is only illustrative of a few particular embodiments with exemplary and/or preferred features, as well as principles of the invention, and that various modifications can be made by those skilled in the art without departing from the scope and spirit of the invention. Therefore, the described embodiments should not be considered as limiting of the scope of the invention in any way. Accordingly, the invention embraces alternatives, modifications and variations which fall within the spirit and scope of the invention as set forth herein and in the claims and any equivalents thereto.

1. A system for disinfecting the surface of a gaming machine, the gaming machine including front and back surfaces, the front surface including a console area, the console area having one or more components intended for user interaction therewith, the system comprising:
a) a lamp mounted adjacent to the console area, the lamp including a housing for supporting an ultraviolet light source therein, the ultraviolet light source being configured to emit ultraviolet light in the range of about 207 nanometers to about 222 nanometers, the housing restricting the transmission of ultraviolet light emitted from the ultraviolet light source in all directions except for in the direction of the one or more components intended for user interaction therewith, wherein illumination of the light source is responsive to detection of a treatment actuating event; and
b) a processing device operatively associated with a memory, the lamp and the gaming machine, wherein the processing device is configured for detecting a treatment actuating event stored in the memory and causing the illumination of the light source responsive to detecting the treatment activating event, wherein the illumination of the light source is deactivated by the processing device responsive to detecting a treatment deactivating event stored in memory.

2. The system as recited in claim 1, wherein the treatment activating event stored in the memory comprises a SAS protocol signal.

3. The system as recited in claim 1, wherein the treatment deactivating event stored in the memory comprises a SAS protocol signal.

4. The system as recited in claim 1, further comprising a motion detector configured to detect the user being positioned adjacent to the console as a treatment deactivating event.

5. The system as recited in claim 1, wherein the housing encloses the ultraviolet light source on all sides except for one side, the one side comprising an opening, opposing side lips and opposing end lips configured to direct ultraviolet light from the ultraviolet light source through the opening and on to the one or more components intended for user interaction.

6. A method for disinfecting the surface of a gaming machine, the gaming machine including front and back surfaces, the front surface including a console area, the console area having one or more components intended for user interaction therewith, the system comprising the steps of:
a) detecting a treatment activating event associated with the gaming machine, wherein the treatment activating event is associated with no user activity at the gaming machine;
b) activating a lamp mounted adjacent to the console area responsive to the detection of the treatment activating event, the lamp including a housing for supporting an ultraviolet light source therein, the ultraviolet light source being configured to emit ultraviolet light in the range of about 207 nanometers to about 222 nanometers, the housing restricting the transmission of ultraviolet light emitted from the ultraviolet light source in all directions except for in the direction of the one or more components intended for user interaction therewith, wherein activating the lamp illuminates the ultraviolet light source;
c) detecting user activity at the gaming machine;
d) deactivating the lamp responsive to the detection of user activity at the gaming machine; and
e) Repeating steps a) through d).

7. The method of claim 6, wherein the step of detecting a treatment activating event further comprises detecting signals generated within the gaming machine through a communication device in communication with the gaming machine.

8. The method of claim 6, wherein the step of detecting a treatment activating event further comprises receiving data from a motion detector configured for detecting motion or the lack thereof in proximity to the gaming machine.
